(19) United States
(12) Patent Application Publication
(10) Pub. No.: US 2026/0060647 A1
(43) Pub. Date: Mar. 5, 2026

(54) METHOD AND SYSTEM FOR SECURING AN ULTRASOUND PROBE OF A WEARABLE ULTRASOUND PROBE SYSTEM TO A PATIENT AT A DESIRED POSITION

(71) Applicant: GE Precision Healthcare LLC, Waukesha, WI (US)

(72) Inventors: Warren Lee, Niskayuna, NY (US); James Sabatini, Glenville, NY (US); Andrew Burns, Niskayuna, NY (US); Weston Griffin, Niskayuna, NY (US); David Shoudy, Niskayuna, NY (US); David Mills, Niskayuna, NY (US)

(21) Appl. No.: 18/821,133

(22) Filed: Aug. 30, 2024

(51) Int. Cl. A61B 8/00

(52) U.S. Cl. CPC A61B 8/4227; A61B 8/4236; A61B 8/4455; A61B 8/461

(57) ABSTRACT

Systems and methods for securing an ultrasound probe of a wearable ultrasound system to a patient at a desired position are provided. The method includes affixing a first side of a pad to a patient or ultrasound probe. The pad includes the first side that is tacky and a second side that is moistened. The second side becomes tacky when moisture is absorbed and/or evaporates. The method includes manipulating the ultrasound probe to the desired position with respect to the patient with the pad between the ultrasound probe and the patient. The second side of the pad that is moistened provides a provisional slidable surface for manipulating the ultrasound probe. The method includes securing a fixture to the ultrasound probe and the patient. The fixture includes a probe attachment portion that is secured to the ultrasound probe and a patient attachment portion that is secured to the patient.

### FIELD

Certain embodiments relate to ultrasound imaging. More specifically, certain embodiments relate to a method and system for securing an ultrasound probe of a wearable ultrasound probe system to a patient at a desired position.

### BACKGROUND

Ultrasound imaging is a medical imaging technique for imaging organs and soft tissues in a human or animal body. Ultrasound imaging uses real time, non-invasive high frequency sound waves to produce a series of two-dimensional (2D), three-dimensional (3D), and/or four-dimensional (4D) (i.e., real-time/continuous 3D images) images.

Ultrasound examinations are typically performed by an ultrasound operator placing an ultrasound transducer on a body surface and manipulating the ultrasound transducer about the body surface to manually control the acquisition of ultrasound image data. The manual manipulation of the ultrasound transducer is not ideal for prolonged ultrasound image data acquisition. Instead, an ultrasound probe, such as a patch probe or the like, may be secured in a fixed position on the body surface of a patient for ultrasound image data acquisition over an extended period of time enabling new continuously or periodically measured diagnostic parameters beyond the acute image parameters. However, in some cases, it may be difficult to identify an optimal position to image an anatomy of interest. Moreover, difficulties may arise in securing the ultrasound probe in place for an extended duration. In addition, providing an acoustic coupling for an extended scan duration can be challenging.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for securing an ultrasound probe of a wearable ultrasound probe system to a patient at a desired position, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system comprising an ultrasound probe of a wearable ultrasound probe system that is operable to be secured to a patient at a desired position, in accordance with various embodiments.

FIG. 2 is a bottom perspective view of an exemplary pad affixed to a wearable ultrasound probe, in accordance with various embodiments.

FIG. 3 is a side perspective view of an exemplary pad affixed to a wearable ultrasound probe, in accordance with various embodiments.

FIG. 4 is a top perspective view of an exemplary wearable ultrasound probe secured to a patient with a fixture, in accordance with various embodiments.

FIG. 5 is an exploded view of an exemplary pad affixed to a patient, in accordance with various embodiments.

FIG. 6 is an illustration of an exemplary ultrasound probe being manipulated to a desired position on a pad affixed to a patient, in accordance with various embodiments.

FIG. 7 is an illustration of an exemplary fixture being secured to an ultrasound probe and a patient to secure the ultrasound probe at a desired position with respect to the patient with a pad between the ultrasound probe and the patient, in accordance with various embodiments.

FIG. 8 is an illustration of an exemplary fixture attachment mechanism being applied to a fixture to further secure the fixture to an ultrasound probe and a patient at a desired position with a pad between the ultrasound probe and the patient, in accordance with various embodiments.

FIG. 9 is an exemplary display of a display system providing feedback for repositioning an ultrasound probe until a correct placement of the ultrasound probe is achieved, in accordance with various embodiments.

FIG. 10 is an exemplary display of an integrated display device of an ultrasound probe that provides feedback for repositioning the ultrasound probe, in accordance with various embodiments.

FIG. 11 is an exemplary display of a display device including sequencing light emitting diodes (LEDs) near a perimeter of an ultrasound probe that provides feedback for repositioning the ultrasound probe, in accordance with various embodiments.

FIG. 12 is a side perspective view of an exemplary snap-on assembly for affixing a pad to an ultrasound probe, in accordance with various embodiments.

FIG. 13 is a side perspective view of an exemplary ultrasound probe affixed with a pad by a snap-on assembly, in accordance with various embodiments.

FIG. 14 is an exploded view of an exemplary coupling of a pad to an upper ring of a snap-on assembly by a fastener ring of the snap-on assembly, in accordance with various embodiments.

FIGS. 15A-15D illustrate exemplary steps for attaching a snap-on assembly having a pad to an ultrasound probe, in accordance with various embodiments.

FIGS. 16A-16B illustrate exemplary steps for attaching another embodiment of the snap-on assembly having a pad to an ultrasound probe, in accordance with various embodiments.

FIG. 17 is a flow chart illustrating exemplary steps that may be utilized for securing an ultrasound probe of a wearable ultrasound probe system to a patient at a desired position, in accordance with various embodiments.

### DETAILED DESCRIPTION

Certain embodiments may be found in a method and system for securing an ultrasound probe of a wearable ultrasound probe system to a patient at a desired position. Aspects of the present disclosure have the technical effect of expediting a positioning of the ultrasound probe to acquire a desired image. Certain embodiments have the technical effect of improved acoustic coupling using a pad for an ultrasound scan over an extended period of time. Various embodiments have the technical effect of securely fixing an ultrasound probe in place for an ultrasound scan over an extended period of time. Aspects of the present disclosure provide the technical effect of improved cleanliness and hygiene due to the ultrasound probe not coming into direct contact with a patient during an ultrasound scan.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be standalone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized, and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

It will be understood that, although the terms first, second, etc. may be used herein to describe various elements, these elements should not be limited by these terms. These terms are only used to distinguish one element from another element. Thus, for example, a first element, a first component or a first section discussed below could be termed a second element, a second component or a second section without departing from the teachings of the present disclosure. Similarly, various spatial terms, such as “upper,” “top,” “lower,” “bottom,” “lateral,” “side,” and the like, may be used in distinguishing one element from another element in a relative manner. It should be understood, however, that components may be oriented in different manners, for example a pad may be turned sideways so that its “top” surface is facing horizontally, and its “side” surface is facing vertically, without departing from the teachings of the present disclosure.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode, which can be one-dimensional (1D), two-dimensional (2D), three-dimensional (3D), or four-dimensional (4D), and comprising Brightness mode (B-mode or 2D mode), Motion mode (M-mode), Color Motion mode (CM-mode), Color Flow mode (CF-mode), Pulsed Wave (PW) Doppler, Continuous Wave (CW) Doppler, Contrast Enhanced Ultrasound (CEUS), and/or sub-modes of B-mode and/or CF-mode such as Harmonic Imaging, Shear Wave Elasticity Imaging (SWEI), Strain Elastography, Tissue Velocity Imaging (TVI), Power Doppler Imaging (PDI), B-Flow Color (BFC), Micro Vascular Imaging (MVI), Ultrasound-Guided Attenuation Parameter (UGAP), and the like, such as where the “image” and/or “plane” includes a single beam or multiple beams. In various embodiments, anatomical or physiological parameters may be derived from ultrasound data, such as fetal femur length, cardiac output, blood velocity, and shear wave velocity, among other things.

Furthermore, the term processor or processing unit, as used herein, refers to any type of processing unit that can carry out the required calculations needed for the various embodiments, such as single or multi-core Central Processing Unit (CPU), Accelerated Processing Unit (APU), Graphic Processing Unit (GPU), Digital Signal Processor (DSP), Field Programmable Gate Array (FPGA), System on a Chip (SoC), Application-Specific Integrated Circuit (ASIC), or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming images may be performed using channel combinations that may originate from more than one transmit event (e.g., synthetic aperture techniques).

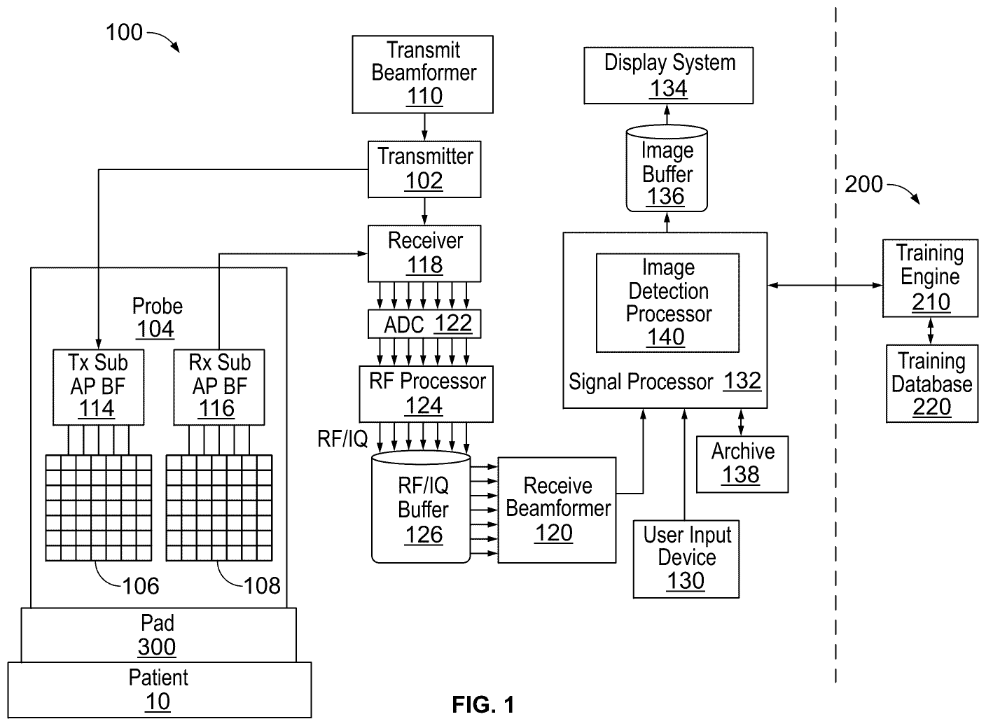

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 comprising an ultrasound probe 104 of a wearable ultrasound probe system 100 that is operable to be secured to a patient 10 at a desired position, in accordance with various embodiments. Referring to FIG. 1, there is shown an ultrasound system 100 and a training system 200. The ultrasound system 100 comprises a transmitter 102, an ultrasound probe 104, a transmit beamformer 110, a receiver 118, a receive beamformer 120, A/D converters 122, a radio frequency (RF) processor 124, a RF/IQ buffer 126, a user input device 130, a signal processor 132, an image buffer 136, a display system 134, and an archive 138.

The transmitter 102 may comprise suitable logic, circuitry, interfaces and/or code that may be operable to drive an ultrasound probe 104. The ultrasound probe 104 may be a wearable ultrasound probe, such as a patch probe or any suitable wearable ultrasound probe, having a phased array, linear array, curved array, or any suitable shape of combination of shapes. The ultrasound probe 104 may be configured to acquire one-dimensional (1D) ultrasound data, two-dimensional (2D) ultrasound data, three-dimensional (3D) ultrasound data, four-dimensional (4D) ultrasound data, and/or any suitable ultrasound data. The ultrasound probe 104 may comprise a group of transmit transducer elements 106 and a group of receive transducer elements 108, that normally constitute the same elements. The group of transmit transducer elements 106 may emit ultrasonic signals through a pad 300 and into a patient 10. In a representative embodiment, the ultrasound probe 104 may be operable to acquire ultrasound image data covering at least a substantial portion of an anatomy, such as a heart or any suitable anatomical structure. In various embodiments, the ultrasound probe 104 may include integrated components, such as visual indicators (e.g., display device, light emitting diodes, etc.), speakers, haptic device(s), and the like.

The ultrasound probe 104 may be acoustically coupled to a patient 10 via a pad 300. The pad 300 may be square, rectangular, circular, oval, and/or any suitable shape. The pad 300 may comprise a first side, a second side, a central portion, and a perimeter portion. In various embodiments, the pad 300 may be tacky unless moistened to provide a slippery surface. The pad 300 may return to tacky after any moisture applied to one or both sides of the pad 300 is absorbed and/or evaporates. In other embodiments, the tackiness may not be moisture dependent. For example, the tackiness may be provided by an adhesive integrated into the pad 300 or adhesive only. In certain embodiments, only the perimeter portion of the pad 300 may be tacky. The pad 300 may be one or a plurality of layers. In certain embodiments, the pad 300 may comprise plurality of wings extending from the perimeter portion of the pad 300. For example, wings may extend from a top of the pad 300, a bottom of the pad 300, and lateral sides of the pad 300 (e.g., 4 wings), or any suitable number and/or configuration of wings. The wings may be a different material than the pad. The wings may be adhesive and may be operable to assist with adhering the pad 300 to the probe 104. In various embodiments, the pad 300, including any wings if present, may include a release liner that may be removed to expose the tacky side of the pad and/or the adhesive of the wings. The pad 300 may comprise hydrogel polymers and/or any suitable flexible porous material. Hydrogel polymers generally comprise an organic cross-linked copolymer, mixed with and plasticized by hydrophilic polymers/oligomers/small molecules. Examples of hydrogel polymers may include N-vinyl pyrolidone/acrylic acid copolymers, polyanilines, polythiophenes (e.g., poly(3,4-ethylenedioxythiophene) (PEDOT), PEDOT doped with polystyrene sulfonate (PSS), hyaluronic acid, cellulose, polyacrylic acid, polyvinyl alcohol, polyurethanes, polyethylene imine, polylactic acid, polylactic-co-glycolic acid, polyacrylamides, polyepsilon-caprolactone, polyethylene glycol (PEG), PEG-acrylates, acrylamides, glycerol, propylene glycol, oligomeric glycols, poly-alcohols, and the like. In various embodiments, the pad 300 may further comprise liquid additives to increase lubricity temporarily (either on their own, in mixtures, or as diluted aqueous mixtures). Depending on the gel composition and the lubricant, some may offer shorter or longer working times. Potential liquid additives my include, for example, glycerol and associated compounds, oligomeric glycols (e.g., polyethylene glycol, polypropylene glycol), oligomeric polyalcohols (e.g., short chain polyvinyl alcohol), ionic surfactants (e.g., sodium lauryl sulfate, sodium dodecyl sulfate, etc.), non-ionic surfactants (e.g., pluronic copolymers), and/or any suitable liquid additives.

In an exemplary embodiment, if the first side of the pad 300 is affixed to a patient 10 and the second side is moistened, the ultrasound probe 104 may be manipulated over the slippery second side of the pad 300 until a desired position is identified. Then, the second side of the pad 300 becomes tacky as the moisture is absorbed and/or evaporates, such that the ultrasound probe 104 becomes affixed to the second side of the pad 300 at the desired position. A fixture (as shown in FIGS. 4, 7, and 8) may then be secured to the ultrasound probe 104 and patient 10 to further secure the ultrasound probe 104 at the desired position with respect to the patient 10, as discussed in more detail below. As another example, if the first side of the pad 300 is affixed to an ultrasound probe 104 and the second side is moistened, the ultrasound probe 104 having the attached pad 300 may be manipulated over the patient 10 with the slippery second side of the pad 300 sliding across the patient 10 until a desired position is identified. Then, the second side of the pad 300 becomes tacky as the moisture is absorbed and/or evaporates, such that the second side of the pad 300 becomes affixed to the patient 10 at the desired position. In other words, the moisture provides a provisional slidable surface. A fixture (as shown in FIGS. 4, 7, and 8) may then be secured to the ultrasound probe 104 and patient 10 to further secure the ultrasound probe 104 at the desired position with respect to the patient 10, as discussed in more detail below. In various embodiments, the fixture may be physically around the ultrasound probe 104 (e.g., loosely attached to the ultrasound probe 104) as the ultrasound probe 104 is manipulated to a desired position and may be securely fixed to the ultrasound probe 104 and the patient 10 once the desired position is obtained. The pad 300 is operable to secure the ultrasound probe 104 at a desired position with respect to the patient 10 for an ultrasound scan over an extended period of time (i.e., hands free), and provide an acoustic coupling for the ultrasound scan over the extended period of time. The pad 300 may provide an additional benefit of improved cleanliness and hygiene since the ultrasound probe 104 may not directly contact the patient 10.

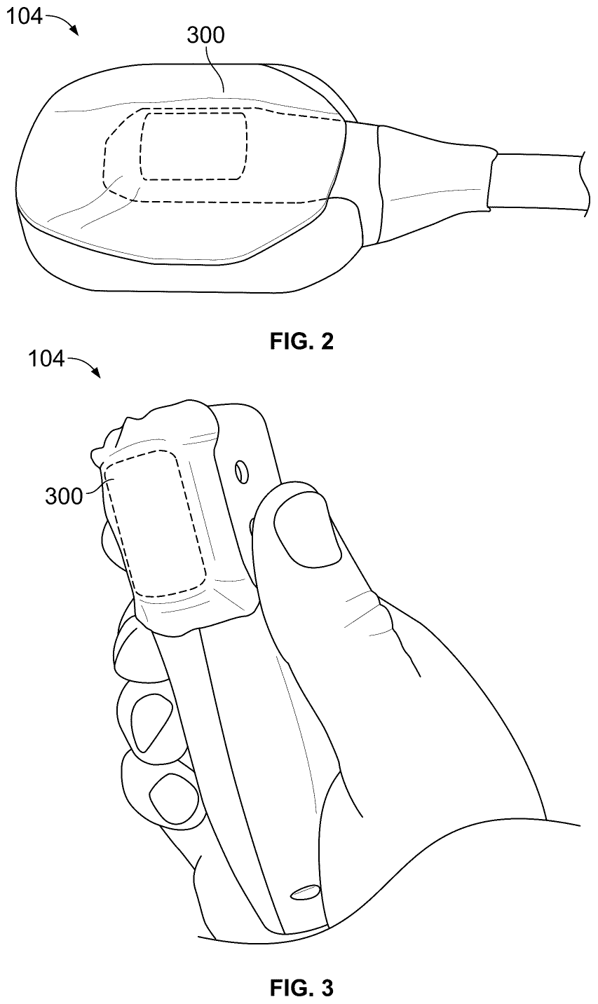

FIG. 2 is a bottom perspective view of an exemplary pad 300 affixed to a wearable ultrasound probe 104, in accordance with various embodiments. FIG. 3 is a side perspective view of an exemplary pad 300 affixed to a wearable ultrasound probe 104, in accordance with various embodiments. Referring to FIGS. 2 and 3, a first side of the pad 300 is affixed to an ultrasound probe 104 (e.g., to a transducer face and the probe housing around the transducer face). The first side of the pad 300 may be tacky for easy coupling to a face of the ultrasound probe 104. A second side of the pad 300 may be moistened to provide a slippery surface that can slide across a patient 10 as the user manipulates the ultrasound probe 104 to a position for acquiring ultrasound images of a desired view over an extended period of time. The second side of the pad 300 may be tacky prior to applying moisture and may become tacky again as the moisture is absorbed and/or evaporates. In other words, the moisture provides a provisional slidable surface. Accordingly, once the ultrasound probe 104 is manipulated to a desired position and the moisture is absorbed and/or evaporates, the second side of the pad 300 becomes affixed to the patient at the desired position.

FIG. 4 is a top perspective view of an exemplary wearable ultrasound probe 104 secured to a patient 10 with a fixture 400, in accordance with various embodiments. Referring to FIG. 4, once the ultrasound probe 104 is manipulated to a desired position on a patient 10 with a pad 300 secured between the ultrasound probe 104 and patient 10, and providing an acoustic coupling between the ultrasound probe 104 and the patient 10, a fixture 400 may be secured to the ultrasound probe 104 and the patient 10 to further fix the ultrasound probe 104 at the desired position for an ultrasound scan over an extended period of time. The fixture 400 may comprise a probe attachment portion 410 and a patient attachment portion 420. The probe attachment portion 410 may be a lockable universal joint and/or any mechanism operable to fixedly attach the fixture 400 to the ultrasound probe 104. The patient attachment portion 420 may be stabilizing supports and/or any mechanism operable to couple to the patient 10. In various embodiments, the fixture 400 may comprise a fixture attachment mechanism 430, such as straps 430, adhesive tape, and/or any suitable mechanism for holding the patient attachment portion 420 against the patient 10 at a fixed position.

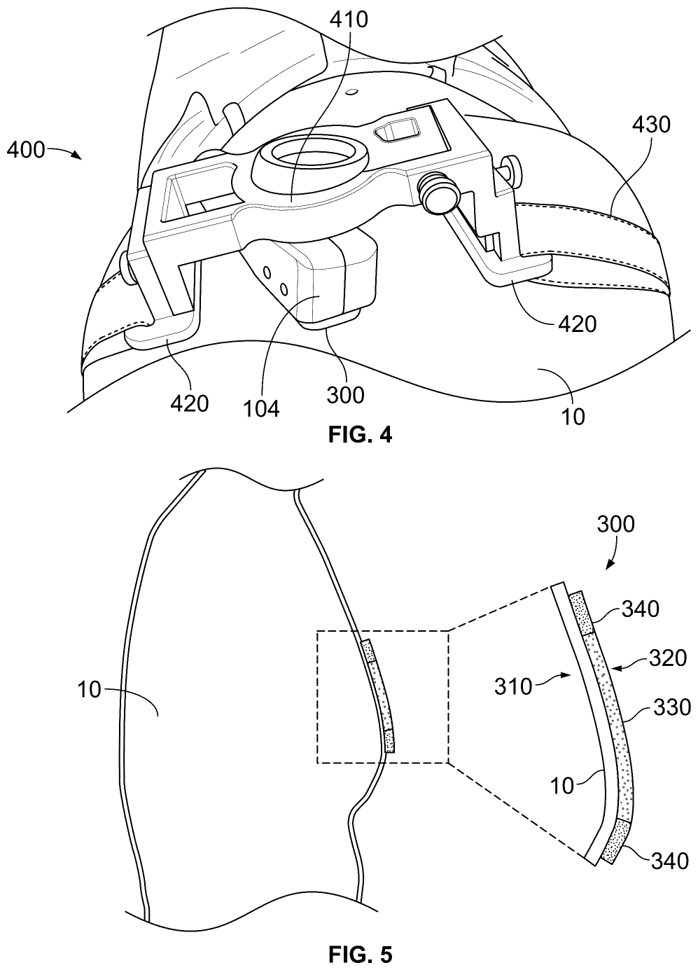

FIG. 5 is an exploded view of an exemplary pad 300 affixed to a patient 10, in accordance with various embodiments. Referring to FIG. 5, a pad 300 affixed to a patient 10 is shown. The pad 300 may comprise a first side 310, a second side 320, a central portion 330, and a perimeter portion 340. The pad 300 may be square, rectangular, circular, oval, and/or any suitable shape. In various embodiments, the pad 300 may be tacky unless moistened to provide a slippery surface. The pad 300 may return to tacky after any moisture applied to one or both sides 310, 320 of the pad 300 is absorbed and/or evaporates. In certain embodiments, only the perimeter portion 340 of the pad 300 may be tacky. As shown in FIG. 5, the first side 310 of the pad 300 is affixed to a patient 10 a second side 320 of the pad 300 is exposed.

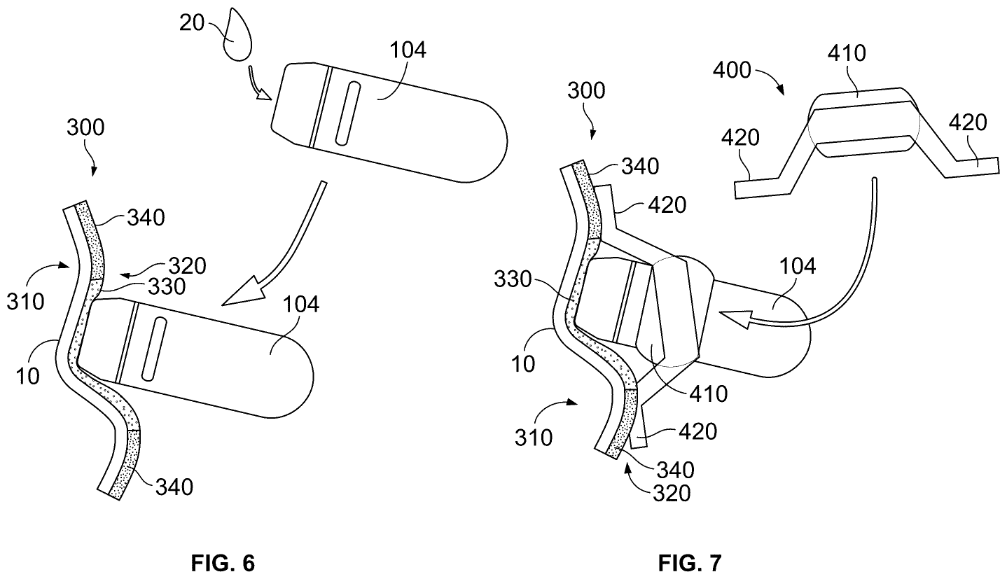

FIG. 6 is an illustration of an exemplary ultrasound probe 104 being manipulated to a desired position on a pad 300 affixed to a patient 10, in accordance with various embodiments. Referring to FIG. 6, a pad 300 affixed to a patient 10 as described above with reference to FIG. 5. After the pad 300 is attached to the patient 10, moisture 20 may be applied to the face of an ultrasound probe 104, or directly to the pad 300, such as in the central portion 330 of the pad 300 on the exposed second side 320. The moisture interacts with the pad 300 to create a slidable surface. The ultrasound probe 104 may be manipulated across the slidable surface of the central portion 330 on the second side 320 of the pad until a position for acquiring a desired image view is obtained. The slidable surface may be temporary (e.g., the moisture 20 provides a provisional slidable surface that becomes tacky once the moisture 20 is absorbed and/or evaporates). In this way, the second side 320 of the pad 300 may become tacky as the moisture is absorbed and/or evaporates, such that the ultrasound probe 104 becomes affixed to the second side 320 of the pad 300 at the desired position.

FIG. 7 is an illustration of an exemplary fixture 400 being secured to an ultrasound probe 104 and a patient 10 to secure the ultrasound probe 104 at a desired position with respect to the patient 10 with a pad 300 between the ultrasound probe 104 and the patient 10, in accordance with various embodiments. Referring to FIG. 7, after a pad 300 is affixed to a patient 10 (as described above with reference to FIG. 5), and an ultrasound probe 104 is manipulated to a desired position on the pad 300 (as described above with reference to FIG. 6), a fixture 400 may be secured to the ultrasound probe 104 and the patient 10 to further fix the ultrasound probe 104 at the desired position for an ultrasound scan over an extended period of time. The fixture 400 may comprise a probe attachment portion 410 and a patient attachment portion 420. The probe attachment portion 410 may be a lockable universal joint and/or any mechanism operable to fixedly attach the fixture 400 to the ultrasound probe 104. The patient attachment portion 420 may be stabilizing supports and/or any mechanism operable to couple to the patient 10. As shown in FIG. 7, the first side 310 of the pad 300 is affixed to a patient 10. The ultrasound probe 104 is positioned against a central portion 330 on a second side 320 of the pad 300. The probe attachment portion 410 of the fixture 400 is coupled to the ultrasound probe 104 and the patient attachment portion 420 of the fixture 400 is coupled to a perimeter portion 340 on the second side 320 of the pad 300. Alternatively, the patient attachment portion 420 of the fixture 400 may be positioned directly on the patient 10. In the embodiment of FIG. 7, the perimeter portion 340 of the pad 300 may be tacky to facilitate a secure coupling of the patient attachment portion 420 of the fixture 400 to the patient 10 via the pad 300.

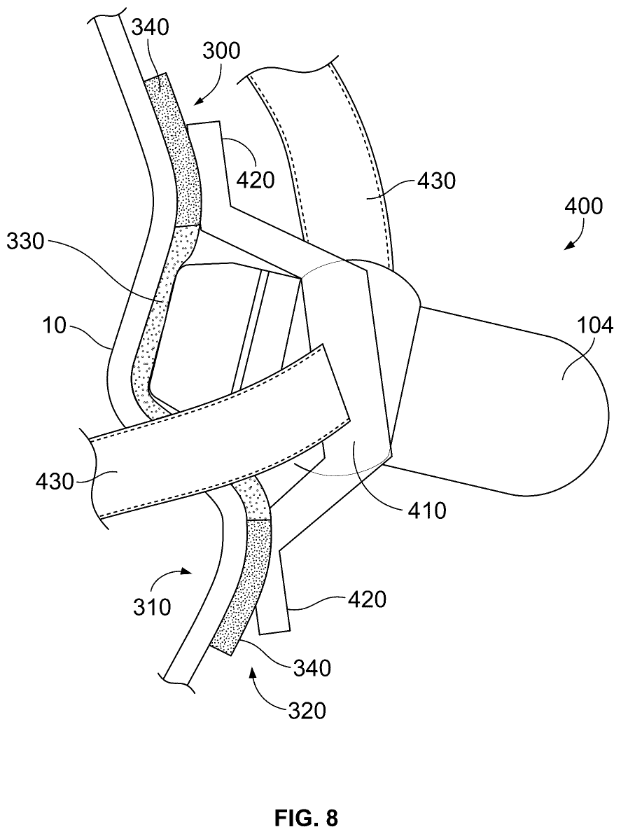

FIG. 8 is an illustration of an exemplary fixture attachment mechanism 430 being applied to a fixture 400 to further secure the fixture 400 to an ultrasound probe 104 and a patient 10 at a desired position with a pad 300 between the ultrasound probe 104 and the patient 10, in accordance with various embodiments. Referring to FIG. 8, after a pad 300 is affixed to a patient 10 (as described above with reference to FIG. 5), an ultrasound probe 104 is manipulated to a desired position on the pad 300 (as described above with reference to FIG. 6), and a fixture 400 is secured to the ultrasound probe 104 and the patient 10 (as described above with reference to FIG. 7), a fixture attachment mechanism 430 may be applied to the fixture 400 to further secure the fixture 400 holding the ultrasound probe 104 to the patient 10 at the desired position for an ultrasound scan over an extended period of time. The fixture attachment mechanism 430 may be straps 430, adhesive tape, and/or any suitable mechanism for holding the patient attachment portion 420 against the patient 10 at a fixed position. As shown in FIG. 8, the first side 310 of the pad 300 is affixed to a patient 10. The ultrasound probe 104 is positioned against a central portion 330 on a second side 320 of the pad 300. The probe attachment portion 410 of the fixture 400 is coupled to the ultrasound probe 104 and the patient attachment portion 420 of the fixture 400 is coupled to a perimeter portion 340 on the second side 320 of the pad 300. The perimeter portion 340 of the pad 300 may be tacky to facilitate a secure coupling of the patient attachment portion 420 of the fixture 400 to the patient 10 via the pad 300. The fixture attachment mechanism 430 (e.g., straps) is coupled to the fixture 400 and the patient 10 to further secure the fixture 400 holding the ultrasound probe 104 to the patient 10 at the desired position for the ultrasound scan over the extended period of time.

Although FIGS. 5-8 illustrate the pad 300 affixed to a patient 10 and the ultrasound probe 104 is manipulated to a desired position on the pad 300 where it is secured, the pad 300 may be flipped such that it is secured to the ultrasound probe 104 instead (similar to the embodiment of FIGS. 2-3 described above). For example, the first side 310 of the pad 300 may be affixed to the ultrasound probe 104 and the second side 320 of the pad 300 may be exposed. Subsequently, moisture 20 may be applied to the patient 10, or directly to the pad 300, such as in the central portion 330 of the pad 300 on the exposed second side 320. The moisture interacts with the pad 300 to create a slidable surface. The ultrasound probe 104 may be manipulated such that the slidable surface of the central portion 330 on the second side 320 of the pad slides across the patient 10 until a position for acquiring a desired image view is obtained. The second side 320 of the pad 300 may become tacky as the moisture is absorbed and/or evaporates, such that the second side 320 of the pad 300 becomes affixed to the patient 10 at the desired position. Then, the fixture 400 may be secured to the ultrasound probe 104 and the patient 10 to further fix the ultrasound probe 104 at the desired position for an ultrasound scan over an extended period of time. Next, the fixture attachment mechanism 430 may be applied to the fixture 400 to further secure the fixture 400 holding the ultrasound probe 104 to the patient 10 at the desired position for an ultrasound scan over an extended period of time.

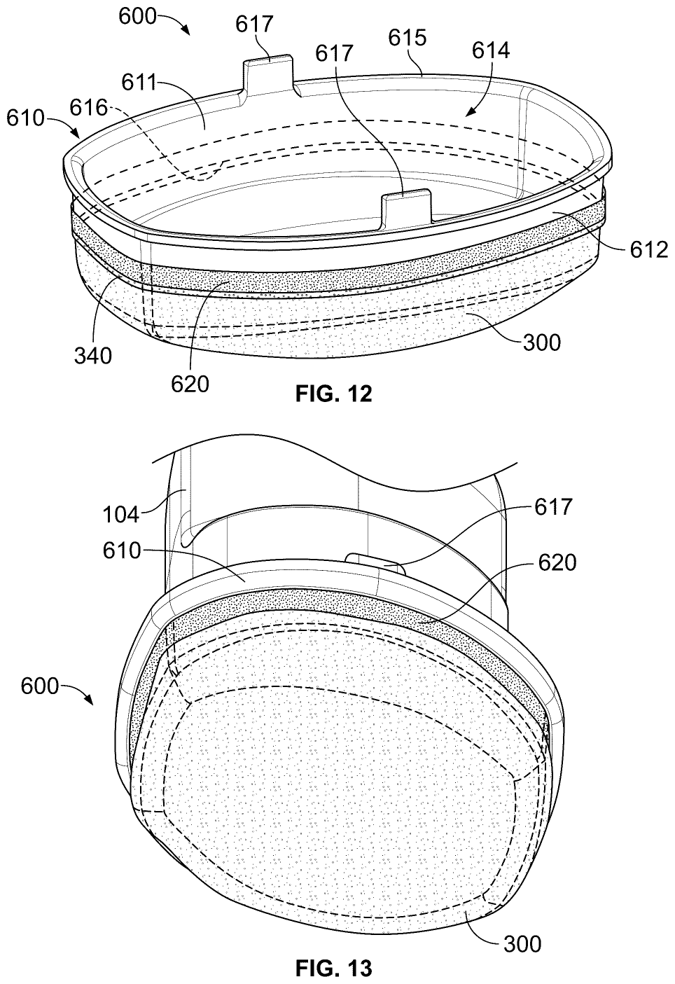

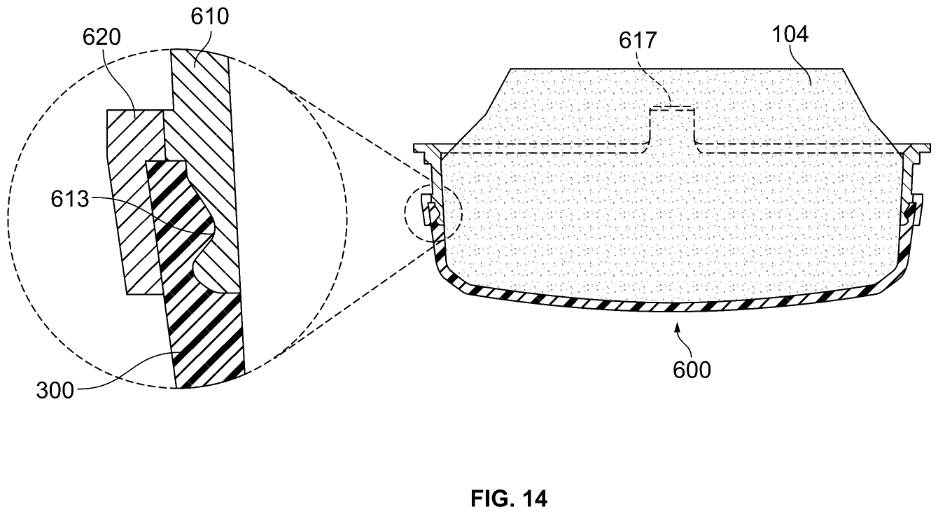

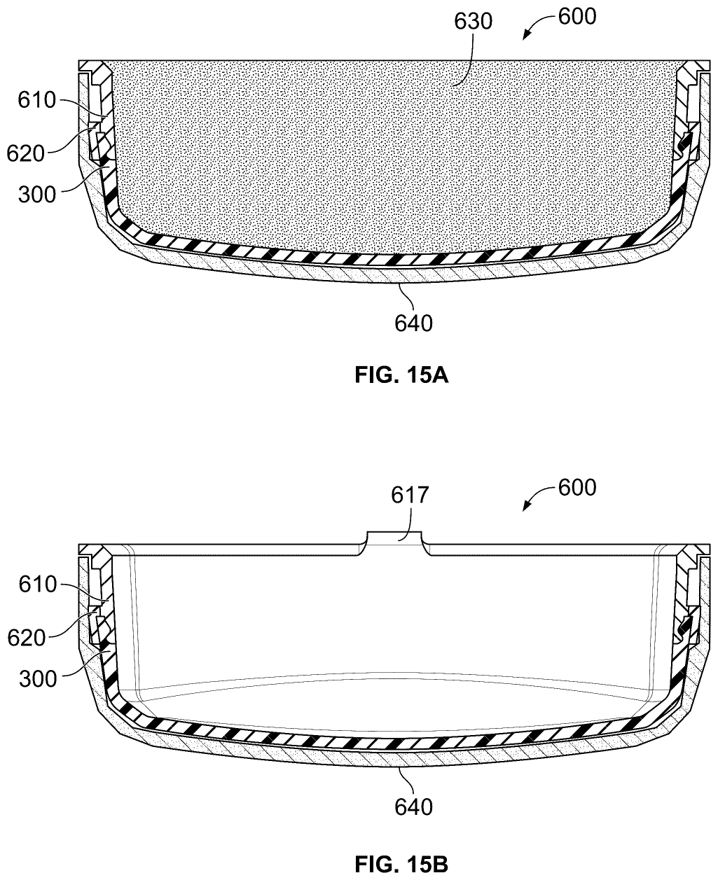

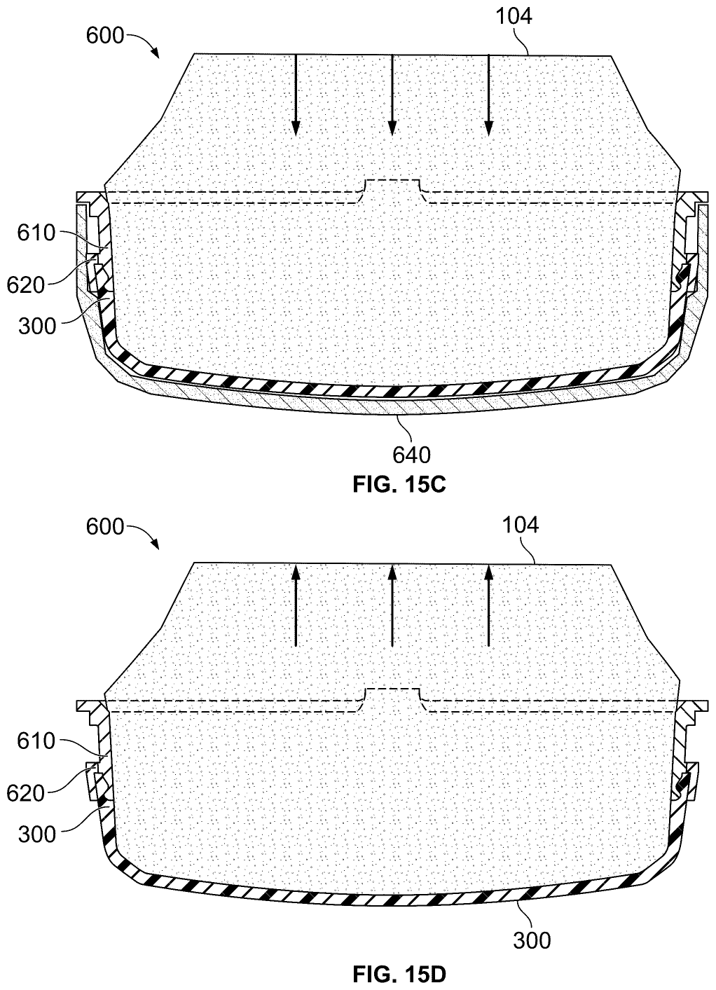

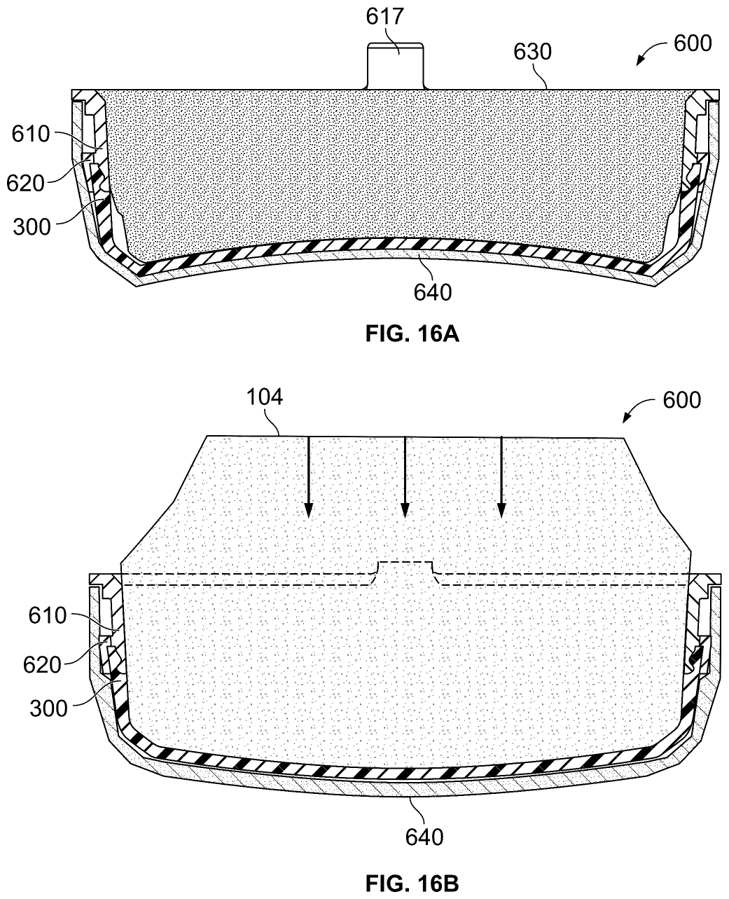

In an exemplary embodiment, the pad 300 may be affixed to the ultrasound probe 104 with a snap-on assembly 600 as shown in FIGS. 12-16. FIG. 12 is a side perspective view of an exemplary snap-on assembly 600 for affixing a pad 300 to an ultrasound probe 104, in accordance with various embodiments. FIG. 13 is a side perspective view of an exemplary ultrasound probe 104 affixed with a pad 300 by a snap-on assembly 600, in accordance with various embodiments. FIG. 14 includes an exploded view (left-most image) of an exemplary coupling of a pad 300 to an upper ring 610 of a snap-on assembly 600 by a fastener ring 620 of the snap-on assembly 600, in accordance with various embodiments. FIGS. 15A-15D illustrate exemplary steps (steps a-d) for attaching a snap-on assembly having a pad to an ultrasound probe, in accordance with various embodiments. FIGS. 16A-16B illustrate exemplary steps (steps a-b) for attaching another embodiment of the snap-on assembly 600 having a pad 300 to an ultrasound probe 104, in accordance with various embodiments. Referring to FIGS. 12-16, the snap-on assembly 600 comprises an upper ring 610, a fastener ring 620, and a pad 300. In various embodiments, the pad 300 may or may not have a pre-formed shape or may be a loosely or tightly stretched film. In certain embodiments, the pad 300 may share various characteristics with the pad 300 described above in connection with any of FIGS. 2, 3, and/or 5-8. The upper ring 610 comprises an inner side 611 and an outer side 612. The inner side 611 of the upper ring 610 is configured to mate with an outer probe surface 104. The outer side 612 may comprise a groove 613. The fastener ring 620 may be operable to press the perimeter portion 340 of the pad 300 into the groove 613 of the upper ring 610 to limit shear stress on the pad 300 to prevent tearing of the pad 300. The upper ring 610 defines an aperture 614 extending through the upper ring 610. The aperture 614 may comprise an upper end 615 and a lower end 616. The upper end 615 may be configured to receive the ultrasound probe 104. The ultrasound probe 104 may extend through the aperture 614, from the upper end 615 and past the lower end 616 to contact a first side of the pad 300. The outer surface of the ultrasound probe 104 mates with the inner side 611 of the upper ring 610. The pad 300 may comprise a perimeter portion 340 affixed to the outer side 612 of the upper ring 610 and covering the lower end 616 of the aperture 614 in the upper ring 610. The fastener ring 620 may be operable to compress the perimeter portion 340 of the pad 300 against the outer side 612 of the upper ring 610. The fastener ring 620 may compress the perimeter portion 340 of the pad 300 into the groove 613 in the outer side 612 of the upper ring 610. In various embodiments, the upper ring 610 may comprise at least one locking tab 617 extending away from the upper ring 610 adjacent the upper end 615 of the aperture 614. The at least one locking tab 617 may be operable to engage with the outer probe surface 104 and/or receive a force in a direction from the inner side 611 to the outer side 612 of the upper ring 610 to disengage the inner side 611 of the upper ring 610 from the outer probe surface 104.

In an exemplary embodiment, the snap-on assembly 600 may be packaged by an inner cup 630 and an outer cup 640. Prior to affixing the pad 300 to the ultrasound probe 104, the snap-on assembly 600 is disposed between the inner cup 630 and the outer cup 640 of removable packaging. The inner cup 630 is removed (see FIGS. 15A-15B) to expose the snap-on assembly 600. The ultrasound probe 104 is attached to the snap-on assembly 600 by inserting the ultrasound probe 104 through the aperture 614 of the upper ring 610 from the upper end 615 to the lower end 616 of the aperture 614 such that the outer probe surface 104 mates with the inner side 611 of the upper ring 610 and an end of the ultrasound probe 104 is pressed into contact with the first side 610 of the pad 300 (see FIG. 15C). The ultrasound probe 104 attached to the snap-on assembly 600 is removed from the outer cup 640 to expose the second side 320 of the pad 300 (see FIG. 15D). In various embodiments, prior to attaching the ultrasound probe 104 to the snap-on assembly 600 (see FIG. 16B), an outer bottom surface of the outer cup 640 comprises a concave profile (see FIG. 16A) to aid in the prevention of air bubbles trapped between the probe face and the first side of the pad. The outer bottom surface of the outer cup 640 is pushed outward into a profile of the ultrasound probe 104 during the attaching the ultrasound probe 104 to the snap-on assembly 600 as the ultrasound probe 104 is pressed into contact with the first side 310 of the pad 300 (see FIG. 16B). In various embodiments, the outer cup 640 may hold moisture 20 against the second side 320 of the pad 300 such that the second side 320 of the pad 300 is pre-wetted when the outer cup 640 is removed.

Referring again to FIG. 1, the transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be backscattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or more of the plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form in-phase and quadrature (IQ) data pairs that are representative of the corresponding echo signals. The RF or IQ signal data may then be communicated to an RF/IQ buffer 126. The RF/IQ buffer 126 may comprise suitable logic, circuitry, interfaces and/or code that may be operable to provide temporary storage of the RF or IQ signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to input patient data, scan parameters, settings, select protocols and/or templates, and the like. In an exemplary embodiment, the user input device 130 may be operable to configure, manage and/or control operation of one or more components, modules, and/or devices in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include button(s), rotary encoder(s), a touchscreen, motion tracking, voice recognition, a mousing device, keyboard, camera and/or any other device capable of receiving a user directive. In certain embodiments, one or more of the user input devices 130 may be integrated into other components, such as the display system 134 or the ultrasound probe 104, for example. As an example, user input device 130 may include a touchscreen display.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), or any suitable device for storing images and related information.

The signal processor 132 may be one or more central processing units, microprocessors, microcontrollers, and/or the like. The signal processor 132 may be an integrated component, or may be distributed across various locations, for example. In an exemplary embodiment, the signal processor 132 may comprise an image detection processor 140. The signal processor 132 may be capable of receiving input information from a user input device 130 and/or archive 138, generating an output displayable by a display system 134, and manipulating the output in response to input information from a user input device 130, among other things. The signal processor 132 and image detection processor 140 may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20 -120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes'worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include an image detection processor 140 that comprises suitable logic, circuitry, interfaces and/or code that may be operable to analyze acquired ultrasound images to determine whether correct placement of the ultrasound probe 104 has been achieved to acquire a desired image view. For example, an ultrasound operator may desire to acquire a particular ultrasound image view of the heart over an extended period of time, such as a four-chamber (4CH) view, two-chamber (2CH) view, apical long-axis (LAX) view, or any suitable view of the heart or any anatomical structure. The ultrasound operator may manipulate an ultrasound probe 104 over a patient 10, with a pad 300 acoustically coupling the probe 104 and patient 10, to navigate to the desired image view. As the ultrasound operator manipulates the ultrasound probe 104, the ultrasound probe is acquiring ultrasound image data, which may be analyzed by the image detection processor 140 to determine whether the desired image view has been obtained. Moreover, the image detection processor 140 may comprise suitable logic, circuitry, interfaces and/or code that may be operable to provide instructions for navigating the ultrasound probe 104 from its current position to a correct position to acquire the desired view.

In this regard, the image detection processor 140 may include, for example, image analysis algorithms, one or more deep neural networks (e.g., a convolutional neural network such as u-net) and/or may utilize any suitable form of image analysis techniques, artificial intelligence, or machine learning processing functionality configured to detect and localize anatomical structures in ultrasound images and/or volumes to determine whether correct placement of the ultrasound probe 104 has been achieved to acquire a desired image view. Additionally and/or alternatively, the image analysis techniques, artificial intelligence, or machine learning processing functionality configured to detect and localize anatomical structures in ultrasound images and/or volumes may be provided by a different processor or distributed across multiple processors at the ultrasound system 100 and/or a remote processor communicatively coupled to the ultrasound system 100. For example, the structure detection and localization functionality may be provided as a deep neural network that may be made up of, for example, an input layer, an output layer, and one or more hidden layers in between the input and output layers. Each of the layers may be made up of a plurality of processing nodes that may be referred to as neurons. For example, the structure detection and localization functionality may include an input layer having a neuron for each pixel of an ultrasound image and/or voxel of an ultrasound volume. The output layer may have a neuron corresponding to each heart muscle, heart chamber, and/or any suitable anatomical structure. Each neuron of each layer may perform a processing function and pass the processed ultrasound image information to one of a plurality of neurons of a downstream layer for further processing. As an example, neurons of a first layer may learn to recognize edges of structure in the obtained ultrasound image and/or volume. The neurons of a second layer may learn to recognize shapes based on the detected edges from the first layer. The neurons of a third layer may learn positions of the recognized shapes relative to landmarks in the obtained ultrasound image and/or volume. The processing performed by the deep neural network may identify anatomical structures and the location of the anatomical structures in the obtained ultrasound images and/or volume with a high degree of probability.

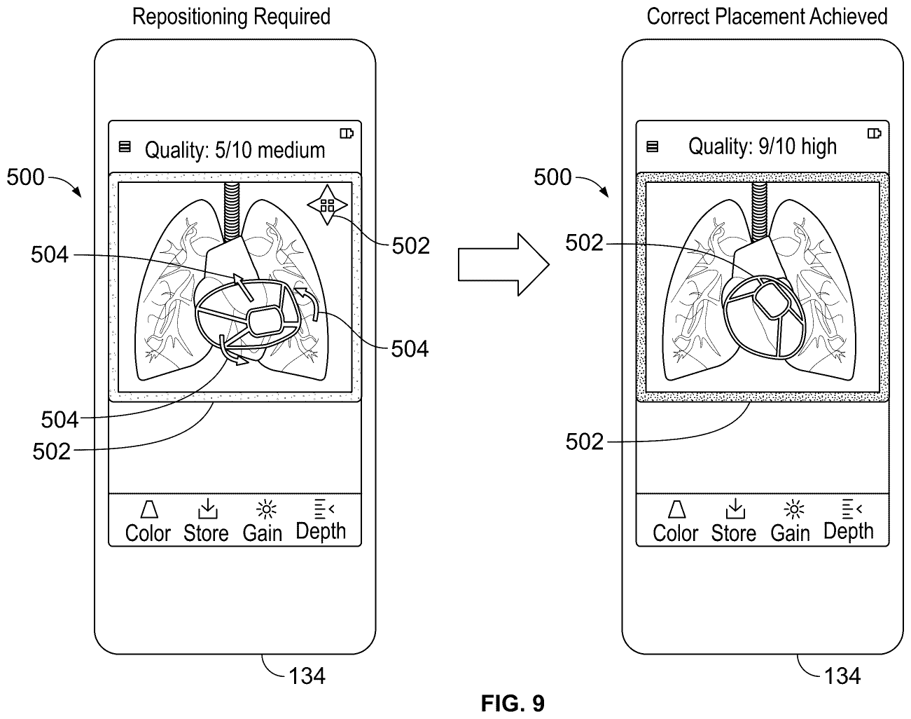

In an exemplary embodiment, the image detection processor 140 may be configured to generate and present probe positioning feedback (as discussed below with respect to FIGS. 9-11) indicating current results of the probe placement (e.g., correct placement or repositioning needed) and/or indicating instructions for positioning the probe correctly. The probe positioning feedback may include visual indicators, audio indicators, and/or haptic feedback. For example, visual probe positioning feedback may comprise pictorial identifiers such as a pictogram and/or structural overlays with markers corresponding with probe placement results and/or probe navigation instructions. Audio positioning feedback may include voice commands, tones, or the like providing instructions for manipulating the ultrasound probe 104 and/or confirmation once proper placement is achieved. Haptic feedback may include sequences of probe vibrations corresponding with probe manipulation directions and/or confirming a correct placement, for example. The probe placement results may include text, icons, audio, haptic feedback and/or the like for indicating correct and/or incorrect placement of the probe 104. The probe navigation instructions may include text, directional icons, audio, haptic feedback and/or the like providing feedback to an operator for manipulating a position and/or orientation of the ultrasound probe 104 and/or adjusting imaging settings to acquire a desired image view. The imaging settings may include gain, depth, zoom level, and/or any suitable image setting.

FIG. 9 is an exemplary display of a display system 134 providing feedback 500, 502, 504 for repositioning an ultrasound probe 104 until a correct placement of the ultrasound probe 104 is achieved, in accordance with various embodiments. Referring to FIG. 9, the display of the display system 134 may provide probe positioning feedback 500 comprising probe placement results 502 and probe navigation instructions 504. The probe placement results 502 may include colors (e.g., yellow if repositioning required or green if correct placement achieved), icons, animations, text, or the like indicating that either repositioning is required, or a correct placement is achieved. The probe navigation instructions 504 may include arrows, text, animations, or the like providing instructions for manipulating the ultrasound probe 104 to the correct placement. For example, if repositioning is required, a yellow box 502 may be provided around the display and an icon 502 symbolizing a need to reposition the probe may be presented along with arrows 504 for instructing a user how to move the probe to a correct position. As another example, once a correct placement is achieved, the yellow box may turn green, the icon 502 symbolizing the need for repositioning may be removed or replaced by a different icon symbolizing correct placement, and the repositioning instructions 504 may be removed, among other things. In various embodiments, a representation of the ultrasound probe 104, a pictogram of the desired view, the currently acquired image, and/or the like may be provided on the display of the display system 134.

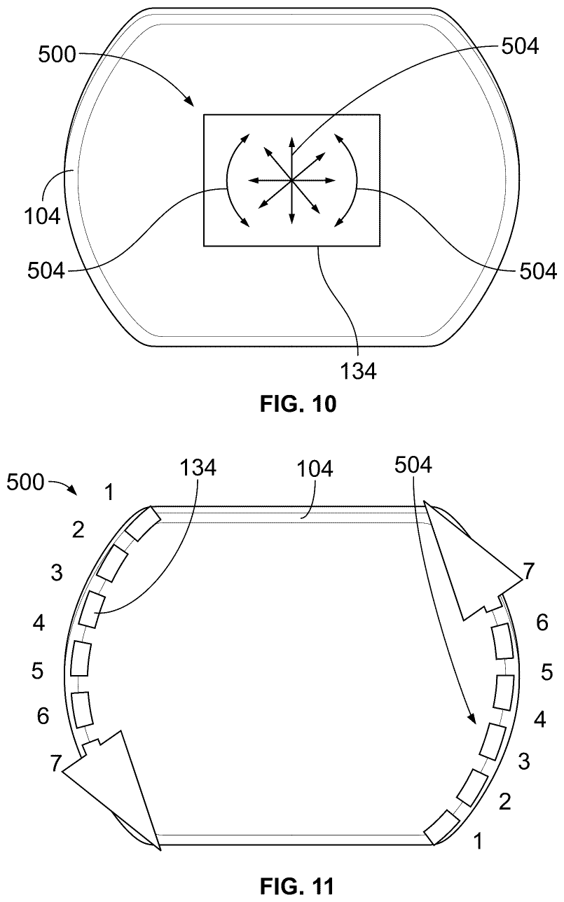

FIG. 10 is an exemplary display of an integrated display device 134 of an ultrasound probe 104 that provides feedback 500, 504 for repositioning the ultrasound probe 104, in accordance with various embodiments. Referring to FIG. 10, the display of the integrated display device 134 may provide probe positioning feedback 500 comprising probe navigation instructions 504. The probe navigation instructions 504 may include arrows, text, animations, or the like providing instructions for manipulating the ultrasound probe 104 to the correct placement. For example, if repositioning is required, arrows 504 for instructing a user how to move the probe to a correct position may be presented. As another example, once a correct placement is achieved, an icon symbolizing correct placement may replace the repositioning instructions 504.

FIG. 11 is an exemplary display of a display device 134 including sequencing light emitting diodes (LEDs) near a perimeter of an ultrasound probe 104 that provides feedback 500, 504 for repositioning the ultrasound probe 104, in accordance with various embodiments. Referring to FIG. 11, the display of the display device 134 may be LEDs that light up in a sequence to provide probe positioning feedback 500 comprising probe navigation instructions 504. The probe navigation instructions 504 may be conveyed by sequentially lighting particular LEDs in particular directions for manipulating the ultrasound probe 104 to the correct placement. For example, if repositioning is required, LEDs on the right side may be sequentially lit in a direction from 1-7 and/or LEDs on the left side may be sequentially lit in a direction from 7-1 to instruct a user to move the ultrasound probe 104 up. As another example, if repositioning is required, LEDs on the left side may be sequentially lit in a direction from 1-7 and/or LEDs on the right side may be sequentially lit in a direction from 7-1 to instruct a user to move the ultrasound probe 104 down. As another example, if repositioning is required, LEDs in a direction from 1-7 may be sequentially lit on both sides to instruct a user to rotate the ultrasound probe 104 counterclockwise. As another example, if repositioning is required, LEDs in a direction from 7-1 may be sequentially lit on both sides to instruct a user to rotate the ultrasound probe 104 clockwise. As another example, if repositioning is required, all LEDs on the left side may be simultaneously lit followed by all LEDs on the right side may be simultaneously lit to instruct a user to translate the ultrasound probe 104 to the right. As another example, if repositioning is required, all LEDs on the right side may be simultaneously lit followed by all LEDs on the left side may be simultaneously lit to instruct a user to translate the ultrasound probe 104 to the left. As another example, once a correct placement is achieved, the LEDs may all be lit or the LEDs may simultaneously flash, among other things.

Referring again to FIG. 1, the display system 134 may be any device capable of communicating visual information to a user. For example, a display system 134 may include a liquid crystal display, a light emitting diode display, and/or any suitable display or displays. The display system 134 can be operable to present ultrasound images, probe positioning feedback 500, 502, 504, and/or any suitable information. The display system 134 may be standalone monitors and/or integrated with various components, such as the user input device 130 (e.g., touchscreen display) or the ultrasound probe 104.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores ultrasound images, instructions for detecting an image view, and/or instructions for providing probe positioning feedback 500, such as probe placement results 502 and/or probe navigation instructions 504, for example.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms. For example, the display system 134 and the user input device 130 may be integrated as a touchscreen display. As another example, the display system 134, speaker(s), haptic device(s), and/or the like may be integrated with the ultrasound probe 104.

Still referring to FIG. 1, the training system 200 may comprise a training engine 210 and a training database 220. The training engine 210 may comprise suitable logic, circuitry, interfaces and/or code that may be operable to train the neurons of the deep neural network(s) (e.g., artificial intelligence model(s)) inferenced (i.e., deployed) by the image detection processor 140. For example, the artificial intelligence model inferenced by the image detection processor 140 may be trained to automatically identify image views using database(s) 220 of classified ultrasound images and/or volumes of different image views. As another example, the artificial intelligence model inferenced by the image detection processor 140 may be trained to automatically determine probe positioning feedback 500, 502, 504 based on locations of target anatomical structures, surrounding anatomical structures, anatomical structure shapes, major/minor axes of anatomical structures, and the like depicted in ultrasound image data using database(s) 220 of classified ultrasound images of possible anatomical structures.

In various embodiments, the databases 220 of training images may be a Picture Archiving and Communication System (PACS), or any suitable data storage medium. In certain embodiments, the training engine 210 and/or training image databases 220 may be remote system(s) communicatively coupled via a wired or wireless connection to the ultrasound system 100 as shown in FIG. 1. Additionally and/or alternatively, components or all of the training system 200 may be integrated with the ultrasound system 100 in various forms.

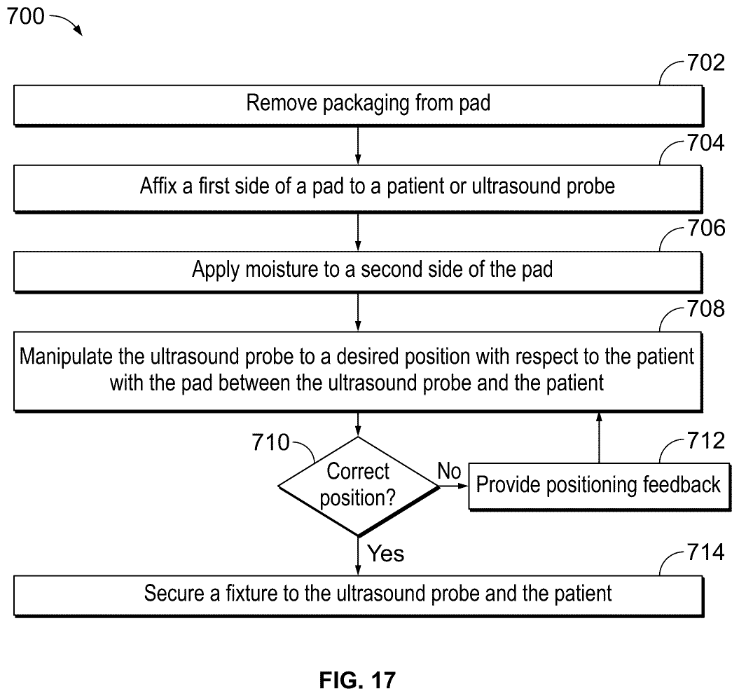

FIG. 17 is a flow chart 700 illustrating exemplary steps 702-714 that may be utilized for securing an ultrasound probe 104 of a wearable ultrasound probe system 100 to a patient 10 at a desired position, in accordance with various embodiments. Referring to FIG. 17, there is shown a flow chart 700 comprising exemplary steps 702 through 714. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 702, packaging 630, 640 may be removed from a pad 300. For example, a first backing sticker may be removed from a first side 310 of a pad 300 prior to affixing the pad 300 to a first of a patient 10 or an ultrasound probe 104, as discussed below with respect to step 704. The removal of the first backing sticker may expose the first side 310 of the pad, which may be tacky. Prior to applying moisture at step 706 (if performed) or prior to manipulating the ultrasound probe to a desired position at step 708, as discussed below, a second backing sticker may be removed from a second side 320 of the pad 300. As another example, various embodiments may provide a snap-on assembly 600 for securing a first side 310 of a pad 300 against an ultrasound probe 104 and exposing a second side 320 of the pad 300 for slidable contact against a patient 10 while the second side 320 of the pad 300 is moist. The second side 320 of the pad 300 may become tacky when the moisture absorbs and/or evaporates to secure the second side 320 of the pad 300 to the patient 10. In such embodiments, the snap-on assembly 600 may comprise packaging 630, 640 that is removed from the pad 300. The packaging 630, 640 may include an inner cup 630 that is removed from an aperture 614 of an upper ring 610 of the snap-on assembly 600 prior to inserting an ultrasound probe 104 through the aperture 614 from an upper end 615 of the aperture and past the lower end 616 of the aperture such that an outer surface of the ultrasound probe 104 mates with an inner surface of the upper ring 610 of the snap-on assembly 600, and the end of the ultrasound probe 104 is pressed against the first side 310 of the pad 300. The ultrasound probe 104 with the attached snap-on assembly 600 may then be pulled away from an outer cup 640 of the packaging 630, 640 to dislodge the ultrasound probe 104 with the snap-on assembly 600 from the outer cup 640 of the packaging 630, 640, thereby exposing the second side 320 of the pad 300. In various embodiments, the outer cup 640 may hold moisture 20 against the second side 320 of the pad 300 such that the second side 320 of the pad 300 is pre-wetted when the outer cup 640 is removed. In certain embodiments, the pad 300 may not be individually packaged and step 702 may be omitted.

At step 704, a first side 310 of the pad 300 may be affixed to a patient 10 or an ultrasound probe 104. For example, the first side 310 of the pad 300 may have a tacky surface that may be stuck against a face of the ultrasound probe 104 or the patient 10. In various embodiments, the first side 310 of the pad 300 may be attached to either of the patient 10 or the ultrasound probe 104.

At step 706, moisture 20 may be applied to a second side 320 of the pad 300. For example, prior to wetting, the second side 320 of the pad 300 may be tacky like the first side 310 of the pad 300. However, the second side 320 of the pad 300 may become slippery when moisture 20 is applied such that the second side 320 of the pad may slide across either the face of the ultrasound probe 104 or the patient 10, depending on which of the ultrasound probe 104 and the patient 10 is coupled to the first side 310 of the pad 300. The second side 320 of the pad may become tacky again when the moisture 20 is absorbed and/or evaporates from the second side 320 of the pad 300. In various embodiments, the second side 320 of the pad 300 may be pre-wetted by packaging 630, 640 of the pad 300 such that the second side 320 is slippery after removal of the packaging 630, 640 from the second side 320. Accordingly, the second side 320 of the pad 300 may be wetted by packaging 630, 640, a user, or in any suitable manner. In an exemplary embodiment, the second side 320 of the pad 300 may have moisture 20 reapplied if the moisture 20 is absorbed and/or evaporates making the second side 320 tacky when a user still desires a slippery surface.

At step 708, the ultrasound probe 104 may be manipulated to a desired position with respect to the patient 10 with the pad 300 between the ultrasound probe 104 and the patient 10. For example, an ultrasound operator may desire to acquire a particular ultrasound image view of the heart over an extended period of time, such as a four-chamber (4CH) view, two-chamber (2CH) view, apical long-axis (LAX) view, parasternal, or any suitable view of the heart or any anatomical structure. The ultrasound operator may manipulate an ultrasound probe 104 over a patient 10, with a pad 300 acoustically coupling the probe 104 and patient 10, to navigate to the desired image view. As an example, if the first side 310 of the pad 300 is affixed to a patient 10 and the second side 320 is moistened, the ultrasound probe 104 may be manipulated over the slippery second side 320 of the pad 300 until a desired position is identified. Then, the second side 320 of the pad 300 becomes tacky as the moisture is absorbed and/or evaporates, such that the ultrasound probe 104 becomes affixed to the second side 320 of the pad 300 at the desired position. As another example, if the first side 310 of the pad 300 is affixed to an ultrasound probe 104 and the second side 320 is moistened, the ultrasound probe 104 having the attached pad 300 may be manipulated over the patient 10 with the slippery second side 320 of the pad 300 sliding across the patient 10 until a desired position is identified. Then, the second side 320 of the pad 300 becomes tacky as the moisture is absorbed and/or evaporates, such that the second side 320 of the pad 300 becomes affixed to the patient 10 at the desired position.

At step 710, a signal processor 132 of the ultrasound system 100 may determine whether the ultrasound probe 104 is in a correct position. For example, as the ultrasound operator manipulates the ultrasound probe 104, the ultrasound probe 104 is acquiring ultrasound image data. An image detection processor 140 of the signal processor 132 of the ultrasound system may be configured to analyze the acquired ultrasound image data to determine whether the desired image view has been obtained. More specifically, the image detection processor 140 may include, for example, image analysis algorithms, one or more deep neural networks (e.g., a convolutional neural network such as u-net) and/or may utilize any suitable form of image analysis techniques, artificial intelligence, or machine learning processing functionality configured to detect and localize anatomical structures in ultrasound images and/or volumes determine whether correct placement of the ultrasound probe 104 has been achieved to acquire a desired image view.

At step 712, if the signal processor 132 of the ultrasound system 100 determines that the ultrasound probe 104 is not in the correct position at step 710, the signal processor 132 may be configured to provide positioning feedback. For example, the image detection processor 140 of the signal processor 132 of the ultrasound system 100 may be configured to generate and present probe positioning feedback 500 indicating current results of the probe placement (e.g., correct placement or repositioning needed) 502 and/or indicating instructions for positioning the probe correctly 504. The probe positioning feedback 500, 502, 504 may include visual indicators, audio indicators, and/or haptic feedback. As an example, visual probe positioning feedback 500 may comprise pictorial identifiers such as a pictogram and/or structural overlays with markers corresponding with probe placement results 502 and/or probe navigation instructions 504. Audio positioning feedback may include voice commands, tones, or the like providing instructions for manipulating the ultrasound probe 104 and/or confirmation once proper placement is achieved. Haptic feedback may include sequences of probe vibrations corresponding with probe manipulation directions and/or confirming a correct placement, for example. The probe placement results 502 may include text, icons, audio, haptic feedback and/or the like for indicating correct and/or incorrect placement of the probe 104. The probe navigation instructions 504 may include text, directional icons, audio, haptic feedback and/or the like providing feedback to an operator for manipulating a position and/or orientation of the ultrasound probe 104 and/or adjusting imaging settings to acquire a desired image view. The imaging settings may include gain, depth, zoom level, and/or any suitable image setting. In various embodiments, the signal processor 132 may additionally and/or alternatively be configured to automatically adjust field of view settings, depth of penetration settings, angle settings, or the like. The signal processor 132 may be configured to provide the updated settings to the ultrasound probe 104 without user intervention.

At step 714, once the ultrasound probe 104 is properly positioned, a fixture 400 may be secured to the ultrasound probe 104 and the patient 10. For example, a fixture 400 may be secured to the ultrasound probe 104 and the patient 10 to further fix the ultrasound probe 104 at the desired position for an ultrasound scan over an extended period of time. The fixture 400 may comprise a probe attachment portion 410 and a patient attachment portion 420. The probe attachment portion 410 may be a lockable universal joint and/or any mechanism operable to fixedly attach the fixture 400 to the ultrasound probe 104. The patient attachment portion 420 may be stabilizing supports and/or any mechanism operable to couple to the patient 10. In various embodiments, the fixture 400 may further comprise a fixture attachment mechanism 430, which may be applied to the fixture 400 to further secure the fixture 400 holding the ultrasound probe 104 to the patient 10 at the desired position for an ultrasound scan over an extended period of time. The fixture attachment mechanism 430 may be straps 430, adhesive tape, and/or any suitable mechanism for holding the patient attachment portion 420 against the patient 10 at a fixed position.

Aspects of the present disclosure provide a method 700 and system 100 for securing an ultrasound probe 104 of a wearable ultrasound probe system 100 to a patient 10 at a desired position. In accordance with various embodiments, the method 700 may comprise affixing 704 a pad 300 to a first of a patient 10 or an ultrasound probe 104. The pad 300 comprises a first side 310 that is tacky and a second side 320 that is moistened 20. The second side 320 becomes tacky when moisture 20 is absorbed and/or evaporates. The first side 310 of the pad is affixed to the first of the patient 10 or the ultrasound probe 104. The method 700 may comprise manipulating 708 the ultrasound probe 104 to the desired position with respect to the patient 10 with the pad 300 between the ultrasound probe 104 and the patient 10. The second side 320 of the pad 300 that is moistened 20 provides a provisional slidable surface for manipulating the ultrasound probe 104. The method 700 may comprise securing 714 a fixture 400 to the ultrasound probe 104 and the patient 10. The fixture 400 comprises a probe attachment portion 410 and a patient attachment portion 420. The probe attachment portion 410 is secured to the ultrasound probe 104 and the patient attachment portion 420 is secured to the patient 10.

In an exemplary embodiment, the second side 320 of the pad 300 comprises a central portion 330 and a perimeter portion 340. The moisture 20 is in the central portion 330 to provide the provisional slidable surface. The perimeter portion 340 is tacky. The patient attachment portion 420 of the fixture 400 is secured to the patient 10 via the perimeter portion 340 of the pad 300. In a representative embodiment, the moisture 20 of the second side 320 of the pad 300 is absorbed and/or evaporates to adhere the second side 320 to a second, opposite the first, of the patient 10 or the ultrasound probe 104. In various embodiments, the method 700 may comprise applying 706 the moisture 20 to the second side 320 of the pad 300 prior to manipulating 708 the ultrasound probe 104. The second side 320 of the pad 300 may be tacky prior to applying the moisture 20 and after the moisture 20 is absorbed and/or evaporates. In certain embodiments, the method 700 may comprise removing 702 a first backing sticker from the first side 310 prior to affixing 704 the pad 300 and removing 702 a second backing sticker from the second side 320 of the pad 300 prior to applying 706 the moisture 20 to the second side 320 of the pad 300.

In a representative embodiment, the affixing 704 the pad 300 to the first of the patient 10 or the ultrasound probe 104 comprises affixing 704 the pad 300 to the ultrasound probe 104 by a snap-on assembly 600 comprising an upper ring 610 and the pad 300. The upper ring 610 may comprise an inner side 611 and an outer side 612. The inner side 611 of the upper ring 610 is configured to mate with an outer probe surface 104. The upper ring 610 defines an aperture 614 extending through the upper ring 610. The aperture 614 may comprise an upper end 615 and a lower end 616. The upper end 615 may be configured to receive the ultrasound probe 104. The pad 300 may comprise a perimeter portion 340 affixed to the outer side 612 of the upper ring 610 and covering the lower end 616 of the aperture 614 in the upper ring 610. In an exemplary embodiment, the snap-on assembly 600 comprises a fastener ring 620 operable to compress the perimeter portion 340 of the pad 300 against the outer side 612 of the upper ring 610. In various embodiments, the outer side 612 of the upper ring 610 comprises a groove 613. The fastener ring 620 may compresses the perimeter portion 340 of the pad 300 into the groove 613 in the outer side 612 of the upper ring 610. In certain embodiments, the upper ring 610 comprises at least one locking tab 617 extending away from the upper ring 610 adjacent the upper end 615 of the aperture 614. The at least one locking tab 617 may be operable to engage with the outer probe surface 104 and/or receive a force in a direction from the inner side 611 to the outer side 612 of the upper ring 610 to disengage the inner side 611 of the upper ring 610 from the outer probe surface 104. In an exemplary embodiment, prior to the affixing 704 the pad 300 to the ultrasound probe 104, the snap-on assembly 600 is disposed between an inner cup 630 and an outer cup 640 of removable packaging. The affixing 704 the pad 300 to the ultrasound probe 104 comprises removing the inner cup 630 to expose the snap-on assembly 600. The affixing 704 the pad 300 to the ultrasound probe 104 comprises attaching the ultrasound probe 104 to the snap-on assembly 600 by inserting the ultrasound probe 104 through the aperture 614 of the upper ring 610 from the upper end 615 to the lower end 616 of the aperture 614 such that the outer probe surface 104 mates with the inner side 611 of the upper ring 610 and an end of the ultrasound probe 104 is pressed into contact with the first side 610 of the pad 300. The affixing 704 the pad 300 to the ultrasound probe 104 comprises removing the ultrasound probe 104 attached to the snap-on assembly 600 from the outer cup 640 to expose the second side 320 of the pad 300. In a representative embodiment, prior to attaching the ultrasound probe to the snap-on assembly, an outer bottom surface of the outer cup 640 comprises a concave profile. The outer bottom surface of the outer cup 640 is pushed outward into a profile of the ultrasound probe 104 during the attaching the ultrasound probe 104 to the snap-on assembly 600 as the ultrasound probe 104 is pressed into contact with the first side 310 of the pad 300.

In certain embodiments, the method 700 may comprise providing 712 feedback 500, 502, 504, by the ultrasound system 100, for manipulating the ultrasound probe 104 to the desired position with respect to the patient 10. The feedback 500, 502, 504 may be provided at visual indicators 134 integrated on the ultrasound probe 104. The visual indicators 134 comprise a display device and/or light emitting diodes. The feedback 500, 502, 504 may be provided at a display system 134 communicatively coupled to the ultrasound probe 104. The feedback 500, 502, 504 may be provided at a speaker configured to provide audible tones. The feedback 500, 502, 504 may be provided at a haptic device embedded within the ultrasound probe 104 and configured to provide haptic feedback. In various embodiments, the affixing 704 the pad 300 to the first of the patient 10 or the ultrasound probe 104 comprises affixing 704 the first side 310 of the pad 300 to the patient 10. The securing 714 the fixture 400 to the ultrasound probe 104 and the patient 10 comprises securing 714 the patient attachment portion 420 to the second side 320 of the pad 300. In an exemplary embodiment, the securing 714 the fixture 400 to the ultrasound probe 104 and the patient 10 further comprises attaching one or more straps 430 to the fixture 400 and the patient 10 and/or applying an adhesive tape 430 to the fixture 400 and the patient 10.

Various embodiments provide a system 100 for securing an ultrasound probe 104 of a wearable ultrasound probe system 100 to a patient 10 at a desired position. The wearable ultrasound probe system 100 may comprise an ultrasound probe 104, a pad 300, and a fixture 400. The pad 300 may comprise a first side 310 that is tacky and a second side 320 that is moistened 20. The second side 320 becomes tacky when moisture 20 is absorbed and/or evaporates. The first side 310 of the pad 300 is operable to be affixed to a first of a patient 10 or the ultrasound probe 104. The second side 320 of the pad 300 that is moistened 20 provides a provisional slidable surface for manipulating the ultrasound probe 104 with respect to the patient 10. The fixture 400 comprises a probe attachment portion 410 and a patient attachment portion 420. The probe attachment portion 410 is configured to secure to the ultrasound probe 104. The patient attachment portion 420 is operable to secure to the patient 10. The fixture 400 is operable to secure the ultrasound probe 104 at a desired position with respect to the patient 10 with the pad 300 between the ultrasound probe 104 and the patient 10.

In certain embodiments, the moisture 20 of the second side 320 of the pad 300 is absorbed and/or evaporates to adhere the second side 320 to a second, opposite the first, of the patient 10 or the ultrasound probe 104. In an exemplary embodiment, the wearable ultrasound probe system 100 may comprise a snap-on assembly 600 operable to affix the pad 300 to the ultrasound probe 104. The snap-on assembly 600 may comprise an upper ring 610 and the pad 300. The upper ring 610 may comprise an inner side 611 and an outer side 612. The inner side 611 of the upper ring 610 may be configured to mate with an outer probe surface of the ultrasound probe 104. The upper ring 610 may define an aperture 614 extending through the upper ring 610. The aperture 614 may comprise an upper end 615 and a lower end 616. The upper end 615 may be configured to receive the ultrasound probe 104. The pad 300 may comprise a perimeter portion 340 affixed to the outer side 612 of the upper ring 610 and covering the lower end 616 of the aperture 614 in the upper ring 600. In a representative embodiment, the snap-on assembly 600 comprises a fastener ring 620 configured to compress the perimeter portion 340 of the pad 300 against the outer side 612 of the upper ring 610. In various embodiments, the outer side 612 of the upper ring 610 comprises a groove 613. The fastener ring 620 may compress the perimeter portion 340 of the pad 300 into the groove 613 in the outer side 612 of the upper ring 600. In certain embodiments, the upper ring 610 comprises at least one locking tab 617 extending away from the upper ring 610 adjacent the upper end 615 of the aperture 614. The at least one locking tab 617 may be operable to engage with the outer probe surface 104 and/or receive a force in a direction from the inner side 611 to the outer side 612 of the upper ring 610 to disengage the inner side 611 of the upper ring 610 from the outer probe surface 104.

In an exemplary embodiment, the wearable ultrasound probe system 100 comprises removable packaging 630, 640 operable to store the snap-on assembly 600 prior to use. The removable packaging 630, 640 comprises an inner cup 630 and an outer cup 640. The snap-on assembly 600 may be disposed between the inner cup 630 and the outer cup 640 prior to use. The inner cup 630 may be removable to expose the snap-on assembly 600. After removal of the inner cup 630, the outer cup 640 may be operable to hold the snap-on assembly 600 until the snap-on assembly 600 is attached to the ultrasound probe 104 by insertion of the ultrasound probe 104 through the aperture 614 of the upper ring 610 from the upper end 615 to the lower end 616 of the aperture 614 such that the outer probe surface 104 is mated with the inner side 611 of the upper ring 600 and an end of the ultrasound probe 104 is pressed into contact with the first side 310 of the pad 300, and the ultrasound probe 104 attached to the snap-on assembly 600 is pulled away from the outer cup 640 to expose the second side 320 of the pad 300.

In a representative embodiment, an outer bottom surface of the outer cup 640 is configured to move outward from a concave profile prior to attachment of the ultrasound probe 104 to the snap-on assembly 600 to a profile of the ultrasound probe 104 after the ultrasound probe 104 attachment to the snap-on assembly 600. The ultrasound probe 104 insertion force flexes the outer cup 640 pushing it into a profile conforming with the probe 104 (i.e. a convex profile). While doing this, the pad 300 within the outer cup 640 first makes contact with the central region of the probe 104. Then, as the outer cup 640 is pressed outward, the pad 300 contact area increases as the outer cup 640 continues to push outward conforming to the probe shape until the probe 104 has been fully inserted and the outer cup 640 has fully popped out assuming the probe shape, and in this process attaching the pad 300 in a manner as to minimize the possibility of air bubbles.

In various embodiments, the wearable ultrasound probe system 100 comprises visual indicators 134, a display system 134, a speaker, and/or a haptic device. The visual indicators 134 may be integrated on the ultrasound probe 104. The visual indicators 134 may comprise a display device 134 and/or light emitting diodes 134 configured to present feedback 500, 502, 504 for manipulating the ultrasound probe 104 to the desired position with respect to the patient 10. The display system 134 may be communicatively coupled to the ultrasound probe 104. The display system 134 may be configured to present the feedback 500, 502, 504 for manipulating the ultrasound probe 104 to the desired position with respect to the patient 10. The speaker may be configured to output audible tones to provide the feedback for manipulating the ultrasound probe 104 to the desired position with respect to the patient 10. The haptic device may be embedded within the ultrasound probe 104 and configured to vibrate to provide the feedback for manipulating the ultrasound probe 104 to the desired position with respect to the patient. In an exemplary embodiment, the wearable ultrasound probe system 100 may comprise one or more straps 430 attached to the fixture 400. The straps 430 are operable to secure the fixture 400 to the patient 10. The wearable ultrasound probe system 100 may comprise an adhesive tape 430 attached to the fixture 400 and the patient 10. The adhesive tape 430 may be operable to secure the fixture 400 to the patient 10.

Certain embodiments provide a system 100 for securing an ultrasound probe 104 of a wearable ultrasound probe system 100 to a patient 10 at a desired position. The wearable ultrasound probe system 100 may comprise an ultrasound probe 104, a snap-on assembly 600, and a fixture 400. The snap-on assembly 600 may be operable to affix a pad 300 to the ultrasound probe 104. The snap-on assembly 600 may comprise an upper ring 610 and the pad 300. The upper ring 610 may comprise an inner side 611 and an outer side 612. The inner side 611 of the upper ring 610 is configured to mate with an outer probe surface of the ultrasound probe 104. The upper ring 610 defines an aperture 614 extending through the upper ring 610. The aperture 614 may comprise an upper end 615 and a lower end 616. The upper end 615 may be configured to receive the ultrasound probe 104. The pad 300 may comprise a perimeter portion 340 affixed to the outer side 612 of the upper ring 610 and covering the lower end 616 of the aperture 614 in the upper ring 610. The pad 300 may comprise a first side 310 and a second side 320 that is moistened 20. The second side 320 may become tacky when moisture 20 is absorbed and/or evaporates. The first side 310 of the pad 300 may be operable to be affixed to the ultrasound probe 104. The second side 320 of the pad 300 that is moistened 20 may provide a provisional slidable surface for manipulating the ultrasound probe 104 against a patient 10. The moisture 20 of the second side 320 of the pad 300 evaporates to adhere the second side 320 to the patient 10. The fixture 400 may comprise a probe attachment portion 410 operable to secure to the ultrasound probe 104. The fixture 400 may comprise a patient attachment portion 420 operable to couple to the patient 10. The fixture 400 may comprise one or more straps 430 operable to secure the fixture 400 to the patient 10. The ultrasound probe 104 may be secured by the fixture 400 at a desired position with respect to the patient 10 with the pad 300 between the ultrasound probe 104 and the patient 10.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for securing an ultrasound probe of a wearable ultrasound probe system to a patient at a desired position.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

1. A method for securing an ultrasound probe of an ultrasound system to a patient at a desired position, the method comprising:
affixing a pad to a first of a patient or an ultrasound probe, wherein the pad comprises a first side that is tacky and a second side that is moistened, wherein the second side becomes tacky when moisture is absorbed and/or evaporates, and wherein the first side of the pad is affixed to the first of the patient or the ultrasound probe;
manipulating the ultrasound probe to the desired position with respect to the patient with the pad between the ultrasound probe and the patient, wherein the second side of the pad that is moistened provides a provisional slidable surface for manipulating the ultrasound probe; and
securing a fixture to the ultrasound probe and the patient, wherein the fixture comprises a probe attachment portion and a patient attachment portion, wherein the probe attachment portion is secured to the ultrasound probe and the patient attachment portion is secured to the patient.

2. The method of claim 1, wherein:
the second side of the pad comprises a central portion and a perimeter portion,
the moisture is in the central portion to provide the provisional slidable surface,
the perimeter portion is tacky, and
the patient attachment portion of the fixture is secured to the patient via the perimeter portion of the pad.

3. The method of claim 1, wherein the moisture of the second side of the pad is absorbed and/or evaporates to adhere the second side to a second, opposite the first, of the patient or the ultrasound probe.

4. The method of claim 3, comprising applying the moisture to the second side of the pad prior to manipulating the ultrasound probe, wherein the second side of the pad is tacky prior to applying the moisture and after the moisture is absorbed and/or evaporates.

5. The method of claim 4, comprising removing a first backing sticker from the first side prior to affixing the pad and removing a second backing sticker from the second side of the pad prior to applying the moisture to the second side of the pad.

6. The method of claim 1, wherein the affixing the pad to the first of the patient or the ultrasound probe comprises affixing the pad to the ultrasound probe by a snap-on assembly comprising:
an upper ring comprising an inner side and an outer side, wherein:
the inner side of the upper ring is configured to mate with an outer probe surface; and
the upper ring defines an aperture extending through the upper ring, the aperture comprising an upper end and a lower end, wherein the upper end is configured to receive the ultrasound probe; and
the pad having a perimeter portion affixed to the outer side of the upper ring and covering the lower end of the aperture in the upper ring.

7. The method of claim 6, wherein the snap-on assembly comprises a fastener ring operable to compress the perimeter portion of the pad against the outer side of the upper ring.

8. The method of claim 7, wherein the outer side of the upper ring comprises a groove, and wherein the fastener ring compresses the perimeter portion of the pad into the groove in the outer side of the upper ring.

9. The method of claim 6, wherein the upper ring comprises at least one locking tab extending away from the upper ring adjacent the upper end of the aperture, the at least one locking tab operable to:
engage with the outer probe surface, and/or
receive a force in a direction from the inner side to the outer side of the upper ring to disengage the inner side of the upper ring from the outer probe surface.

10. The method of claim 6, wherein prior to the affixing the pad to the ultrasound probe, the snap-on assembly is disposed between an inner cup and an outer cup of removable packaging, and wherein the affixing the pad to the ultrasound probe comprises:
removing the inner cup to expose the snap-on assembly;
attaching the ultrasound probe to the snap-on assembly by inserting the ultrasound probe through the aperture of the upper ring from the upper end to the lower end of the aperture such that the outer probe surface mates with the inner side of the upper ring and an end of the ultrasound probe is pressed into contact with the first side of the pad; and
removing the ultrasound probe attached to the snap-on assembly from the outer cup to expose the second side of the pad.

11. The method of claim 10, wherein:
prior to attaching the ultrasound probe to the snap-on assembly, an outer bottom surface of the outer cup comprises a concave profile; and
the outer bottom surface of the outer cup is pushed outward into a profile of the ultrasound probe during the attaching the ultrasound probe to the snap-on assembly as the ultrasound probe is pressed into contact with the first side of the pad.

12. The method of claim 1, comprising providing feedback, by the ultrasound system, for manipulating the ultrasound probe to the desired position with respect to the patient, wherein the feedback is provided at one of:
visual indicators integrated on the ultrasound probe, wherein the visual indicators comprise a display device and/or light emitting diodes;
a display system communicatively coupled to the ultrasound probe;
a speaker configured to provide audible tones; or a haptic device embedded within the ultrasound probe and configured to provide haptic feedback.

13. The method of claim 1, wherein:
the affixing the pad to the first of the patient or the ultrasound probe comprises affixing the first side of the pad to the patient; and
the securing the fixture to the ultrasound probe and the patient comprises securing the patient attachment portion to the second side of the pad.

14. The method of claim 1, wherein the securing the fixture to the ultrasound probe and the patient further comprises:
attaching one or more straps to the fixture and the patient, and/or
applying an adhesive tape to the fixture and the patient.

15. A wearable ultrasound probe system comprising:
an ultrasound probe;
a pad comprising a first side that is tacky and a second side that is moistened, wherein the second side becomes tacky when moisture is absorbed and/or evaporates, wherein:
the first side of the pad is operable to be affixed to a first of a patient or the ultrasound probe; and
the second side of the pad that is moistened provides a provisional slidable surface for manipulating the ultrasound probe with respect to the patient; and
a fixture comprising a probe attachment portion and a patient attachment portion, wherein:
the probe attachment portion is configured to secure to the ultrasound probe;
the patient attachment portion is operable to secure to the patient; and
the fixture is operable to secure the ultrasound probe at a desired position with respect to the patient with the pad between the ultrasound probe and the patient.

16. The wearable ultrasound probe of claim 15, wherein the moisture of the second side of the pad is absorbed and/or evaporates to adhere the second side to a second, opposite the first, of the patient or the ultrasound probe.

17. The wearable ultrasound probe system of claim 15, comprising a snap-on assembly operable to affix the pad to the ultrasound probe, the snap-on assembly comprising:
an upper ring comprising an inner side and an outer side, wherein:
the inner side of the upper ring is configured to mate with an outer probe surface of the ultrasound probe; and
the upper ring defines an aperture extending through the upper ring, the aperture comprising an upper end and a lower end, wherein the upper end is configured to receive the ultrasound probe; and
the pad having a perimeter portion affixed to the outer side of the upper ring and covering the lower end of the aperture in the upper ring.

18. The wearable ultrasound probe system of claim 17, wherein the snap-on assembly comprises a fastener ring configured to compress the perimeter portion of the pad against the outer side of the upper ring.

19. The wearable ultrasound probe system of claim 18, wherein the outer side of the upper ring comprises a groove, and wherein the fastener ring compresses the perimeter portion of the pad into the groove in the outer side of the upper ring.

20. The wearable ultrasound probe system of claim 17, wherein the upper ring comprises at least one locking tab extending away from the upper ring adjacent the upper end of the aperture, the at least one locking tab operable to:
engage with the outer probe surface, and/or
receive a force in a direction from the inner side to the outer side of the upper ring to disengage the inner side of the upper ring from the outer probe surface.

21. The wearable ultrasound probe system of claim 15, comprising removable packaging operable to store the snap-on assembly prior to use, the removable packaging comprising an inner cup and an outer cup, wherein:
the snap-on assembly is disposed between the inner cup and the outer cup prior to use;
the inner cup is removable to expose the snap-on assembly;
after removal of the inner cup, the outer cup is operable to hold the snap-on assembly until:
the snap-on assembly is attached to the ultrasound probe by insertion of the ultrasound probe through the aperture of the upper ring from the upper end to the lower end of the aperture such that the outer probe surface is mated with the inner side of the upper ring and an end of the ultrasound probe is pressed into contact with the first side of the pad; and
the ultrasound probe attached to the snap-on assembly is pulled away from the outer cup to expose the second side of the pad.

22. The wearable ultrasound probe system of claim 21, wherein an outer bottom surface of the outer cup is configured to move outward from a concave profile prior to attachment of the ultrasound probe to the snap-on assembly to a profile of the ultrasound probe after the ultrasound probe attachment to the snap-on assembly.

23. The wearable ultrasound probe system of claim 15, comprising:
visual indicators integrated on the ultrasound probe, the visual indicators comprising a display device and/or light emitting diodes configured to present feedback for manipulating the ultrasound probe to the desired position with respect to the patient;
a display system communicatively coupled to the ultrasound probe, the display system configured to present the feedback for manipulating the ultrasound probe to the desired position with respect to the patient;
a speaker configured to output audible tones to provide the feedback for manipulating the ultrasound probe to the desired position with respect to the patient; and/or a haptic device embedded within the ultrasound probe and configured to vibrate to provide the feedback for manipulating the ultrasound probe to the desired position with respect to the patient.

24. The wearable ultrasound probe system of claim 15, comprising:
one or more straps attached to the fixture, wherein the straps are operable to secure the fixture to the patient; and/or
an adhesive tape attached to the fixture and the patient, wherein the adhesive tape is operable to secure the fixture to the patient.

25. A wearable ultrasound probe system comprising:
an ultrasound probe;
a snap-on assembly operable to affix a pad to the ultrasound probe, the snap-on assembly comprising:
an upper ring comprising an inner side and an outer side, wherein:
the inner side of the upper ring is configured to mate with an outer probe surface of the ultrasound probe; and
the upper ring defines an aperture extending through the upper ring, the aperture comprising an upper end and a lower end, wherein the upper end is configured to receive the ultrasound probe; and
the pad having a perimeter portion affixed to the outer side of the upper ring and covering the lower end of the aperture in the upper ring, the pad comprising a first side and a second side that is moistened, wherein the second side becomes tacky when moisture is absorbed and/or evaporates, wherein:
the first side of the pad is operable to be affixed to the ultrasound probe;
the second side of the pad that is moistened provides a provisional slidable surface for manipulating the ultrasound probe against a patient; and
the moisture of the second side of the pad evaporates to adhere the second side to the patient; and
a fixture comprising:
a probe attachment portion operable to secure to the ultrasound probe;
a patient attachment portion operable to couple to the patient; and
one or more straps operable to secure the fixture to the patient,
wherein the ultrasound probe is secured by the fixture at a desired position with respect to the patient with the pad between the ultrasound probe and the patient.
